(19) United States
(12) Patent Application Publication
(10) Pub. No.: US 2024/0382640 A1
(43) Pub. Date: Nov. 21, 2024

(54) Fragrance Delivery System

(71) Applicant: ELC MANAGEMENT LLC, Melville, NY (US)

(72) Inventors: Daniel O. Dedewo, Astoria, NY (US); David Edward Wilson, Plainview, NY (US); Sezen Buell, Harrington Park, NJ (US); Gianluca Mattaroccia, Sunnyside, NY (US); Romain Julien Guillevic, Belz (FR)

(21) Appl. No.: 18/199,692

(22) Filed: May 19, 2023

(51) Int. Cl. A61L 9/03; B29C 64/153; B33Y 80/00

(52) U.S. Cl. CPC A61L 9/03; B29C 64/153; B33Y 80/00; A61L 2209/11; B33Y 10/00

(57) ABSTRACT

Methods of manufacturing a porous device for diffusing a fragrance using additive manufacturing including iteratively spreading a loose powder and selecting at least one process parameter to create a porosity within the porous device adapted to carry the fragrance from a fragrance reservoir to an exterior surface of the porous device. An additively manufactured device for diffusing a fragrance includes a diffuser having a plurality of pores defining a porosity of the diffuser, the porosity defining at least one of a rate of absorption, an amount of storage, and a rate of diffusion of the fragrance. A method of using a porous device includes filling the plurality of pores with fragrance and diffusing the fragrance from an exterior surface.

### FIELD OF THE DISCLOSURE

The present disclosure generally relates to methods for additively manufacturing a device for diffusing a fragrance, the device itself, and methods of using the device. More particularly, the present disclosure relates to additive manufacturing methods wherein at least one process parameter is selected to create a porosity within the device adapted to carry the fragrance from a fragrance reservoir to an exterior surface.

### BACKGROUND

Fragrance diffusers provide a means by which a solid or liquid fragrance can be emitted into the air for a user to enjoy. Simple diffusers that rely on evaporation are relatively inexpensive and do not require access to a power source. For example, reed diffusers have reeds placed within a container of liquid fragrance, and the fragrance travels up from the container along capillaries within the reeds to the tops of the reeds, where the fragrance evaporates. However, the amount of fragrance that can be released by evaporation within a given time (and the corresponding intensity of scent perceived by the user) is limited by the relatively small surface area of the reeds connected to the capillaries. Electronic diffusers are typically able to diffuse a greater amount of fragrance within a given time, thereby increasing the intensity of scent perceived by the user. For example, ultrasonic diffusers use a vibrating diaphragm to turn a solution of water and oil into a fine mist. Nebulizers diffuse concentrated oil by blowing compressed air through it to turn it to mist. However, electronic diffusers are typically more expensive and require either batteries or a connection to a power source.

Accordingly, there is a need for fragrance diffusers having improved functionalities.

### SUMMARY

Examples within the scope of the present disclosure are directed approaches for manufacturing applicators for applying a cosmetic substance.

In an example, a method of manufacturing a porous device for diffusing a fragrance using additive manufacturing includes iteratively spreading a loose powder and depositing a binder on the loose powder in a defined pattern. The defined pattern is configured to contribute to a porosity of the porous device. The method further includes increasing a temperature to cause particle sintering. At least one process parameter is selected to create a porosity within the porous device adapted to carry the fragrance from a fragrance reservoir to an exterior surface of the porous device.

In an approach, the at least one process parameter may be at least one of a thickness of layers of the loose powder, a viscosity, a surface tension, a solid content, a pH level, a drying rate, an adhesion strength of the binder, a shape of the loose powder, an average particle size of the loose powder, a size distribution of the loose powder, a sintering temperature, and a sintering time.

In an approach, the loose powder may comprise at least one of ceramic, metal, glass, and composites.

In an approach, the method may further include saturating the porous device with a fragrance.

In an approach, the method may further include sealing the porous device within an external housing.

In an example, a method of manufacturing a porous device for diffusing a fragrance using additive manufacturing includes iteratively spreading a loose powder to form a powder bed and using a heat source to selectively melt regions of the powder bed based on a 3D model. At least one process parameter is selected to create a porosity within the porous device adapted to carry the fragrance from a fragrance reservoir to an exterior surface of the porous device.

In an approach, the at least one process parameter may be at least one of beam diameter of the heat source, a power of the heat source, a layer thickness, a scanning speed, a powder spreading method, a type of polymer powder, a type of composite powder, a shape of the loose powder, an average particle size of the loose powder, a laser wavelength, a size distribution of the loose powder, and an energy output from an infrared source.

In an approach, the method further may include saturating the porous device with a fragrance.

In an example, an additively manufactured device for diffusing a fragrance includes a diffuser including a plurality of pores. The plurality of pores are configured to absorb, store, and diffuse a fragrance, and the plurality of pores define a porosity of the diffuser. The porosity of the diffuser defines at least one of a rate of absorption, an amount of storage, and a rate of diffusion of the fragrance.

In an approach, the device may include a base having a fragrance reservoir configured to hold the fragrance. The diffuser may include a fragrance distribution structure configured for placement within the fragrance reservoir of the base. The diffuser may further include an exterior surface fluidly connected to the fragrance distribution structure.

In an approach, a plurality of diffusers may be configured for placement within the fragrance reservoir of the base.

In an approach, the base may include a pressure control to create a pressure differential to drive the fragrance from the fragrance reservoir into the fragrance distribution structure and toward the exterior surface.

In an approach, the device may include at least one of an air circulation device and a heating element.

In an example, a method of using a porous device includes providing a porous diffuser having a fragrance distribution structure, a plurality of pores, and an exterior surface. The method further includes providing a base having a fragrance reservoir, filling the fragrance reservoir of the base with a fragrance, and placing the fragrance distribution structure of the porous diffuser within the fragrance in the fragrance reservoir of the base to fill the plurality of pores of the porous diffuser with the fragrance. The method further includes removing the porous diffuser from the base, diffusing the fragrance from the exterior surface of the porous diffuser, and returning the porous diffuser to the base with the fragrance distribution structure within the fragrance reservoir to refill the plurality of pores of the porous diffuser with the fragrance.

In an approach, the method may include refilling the fragrance reservoir with the fragrance.

In an approach, the method may include creating a pressure differential to drive the fragrance from the fragrance reservoir into the fragrance distribution structure and toward the exterior surface.

In an approach, the method may include providing a visual characteristic within the fragrance that is visible within the porous diffuser when the fragrance is in the plurality of pores.

In an approach, the degree to which the visual characteristic within the fragrance is visible at the exterior surface of the porous diffuser correlates with the amount of fragrance present within the porous diffuser.

In an approach, a subset of the plurality of pores are configured to trap the fragrance such that the degree to which the visual characteristic within the fragrance is visible at the exterior surface of the porous diffuser increases with use of the porous diffuser as more of the plurality of pores within the subset trap the fragrance.

In an approach, the porous diffuser has a fragrance repository in fluid communication with the exterior surface via the plurality of pores, the plurality of pores adapted to transmit the fragrance from the fragrance repository to the exterior surface at a target rate.

### BRIEF DESCRIPTION OF THE DRAWINGS

The above needs are at least partially met through provision of one, more than one, or any combination of the approaches described in the following detailed description, particularly when studied in conjunction with the drawings, wherein:

FIG. 1 illustrates a perspective view of an example device for diffusing a fragrance including a diffuser and a base in a closed configuration accordance with various examples.

FIG. 2 illustrates a perspective view of the device of FIG. 1 in an open configuration for filling with a fragrance.

FIG. 3A illustrates a perspective view of the diffuser of the device of FIGS. 1 and 2.

FIG. 3B illustrates an exploded view of pores of the diffuser of FIG. 3A.

FIG. 4A illustrates a perspective view of a first porosity of a diffuser in accordance with various examples.

FIG. 4B illustrates a cross-sectional view of the first porosity of FIG. 4A.

FIG. 5A illustrates a perspective view of a second porosity of a diffuser in accordance with various examples.

FIG. 5B illustrates a cross-sectional view of the second porosity of FIG. 5A.

FIG. 6A illustrates a perspective view of a third porosity of a diffuser in accordance with various examples.

FIG. 6B illustrates a cross-sectional view of the third porosity of FIG. 6A.

FIG. 7 illustrates a perspective view of an example device for diffusing a fragrance including a plurality of diffusers resembling flowers and a single base resembling a vase.

FIG. 8 illustrates a perspective view of an example device for diffusing a fragrance including a plurality of diffusers resembling flowers and a single base resembling a stem positioned in a secondary support resembling a vase.

FIG. 9 illustrates a cross-sectional view of an example device for diffusing a fragrance including a plurality of diffusers resembling flower petals and a single base resembling a carpel positioned in a secondary support resembling a stem.

FIG. 10 illustrates a method of using a porous fragrance diffuser in accordance with various examples.

FIG. 11 illustrates a method of additively manufacturing a porous fragrance diffuser using particle sintering in accordance with various examples.

FIG. 12 illustrates a method of additively manufacturing a porous fragrance diffuser by selectively melting regions of a powder bed using a heat source in accordance with various examples.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted in order to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various approaches, a fragrance diffuser is provided that can be manufactured to have a porosity that optimizes the user's experience in providing, storing, or diffusing a chosen fragrance. For example, the fragrance diffuser may be associated with a selected rate of absorption, amount of storage, and/or rate of diffusion of the fragrance. The porosity of the fragrance diffuser is achieved by additive manufacturing. In contrast to typical additive manufacturing techniques that seek to maximize the density of material distributed, the disclosed methods of manufacturing involve intentionally selecting at least one process parameter in order to create a desired porosity. Using these techniques, pore dimensions and frequency can be determined based on the specific fragrance being diffused with, for example, smaller pores and/or less frequent pores for lower viscosity fragrances and larger pores and/or more frequent pores for higher viscosity fragrances. A porous diffuser can take a variety of forms, including wearables, house ornaments, and car accessories. The porous diffuser can be placed in a base to absorb the fragrance, worn or otherwise used throughout the day, and returned to the base in the evening.

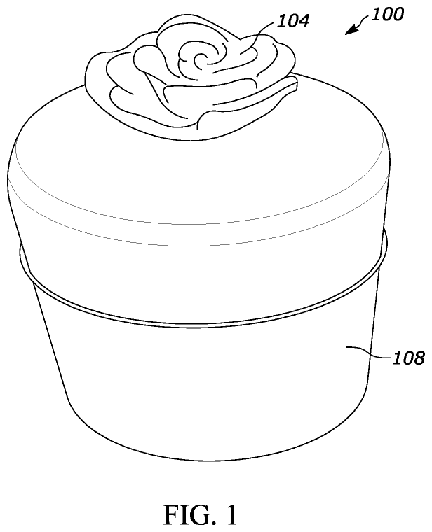

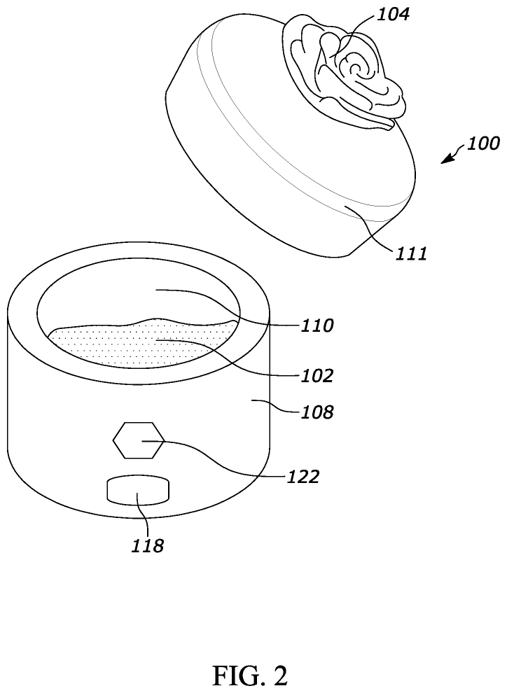

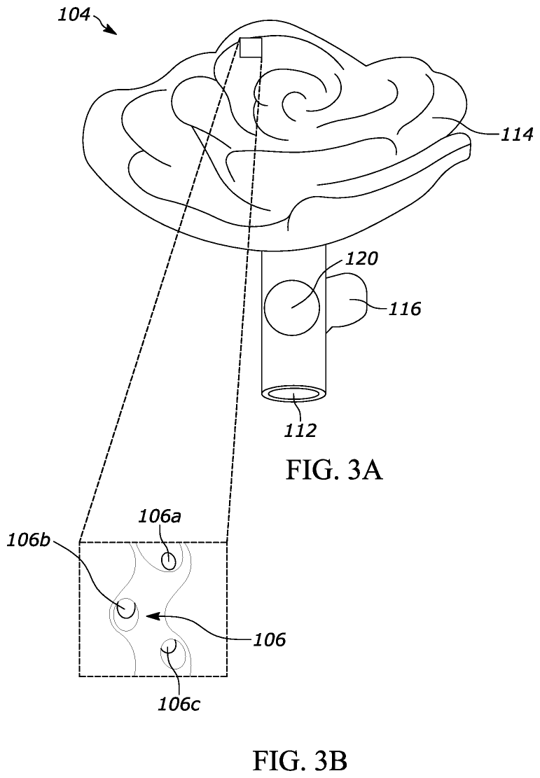

FIGS. 1 and 2 illustrates an additively manufactured device 100 for diffusing a fragrance 102 (shown in FIG. 2). The device 100 includes a diffuser 104 that, in the arrangement shown in FIGS. 1, 2, and 3A, is shaped like a flower. In other arrangements, the diffuser 104 may have a different ornamental or aesthetic appearance depending on the intended use and stylistic preferences. As shown in FIG. 3B, the diffuser 104 includes a plurality of pores 106 (106a, 106b, 106c). The plurality of pores 106 are configured to absorb, store, and diffuse the fragrance 102. The porosity of the diffuser 104 defines at least one of a rate of absorption, an amount of storage, and a rate of diffusion of the fragrance 102.

As shown in FIG. 2, the device 100 includes a base 108 having a fragrance reservoir 110 configured to hold the fragrance 102. The base 108 shown in FIG. 2 includes a refill mechanism 111 that is a lid configured to be removed to allow the fragrance reservoir 110 to be refilled. In other arrangements, the refill mechanism 111 may be an aperture or another structure allowing the fragrance reservoir 110 to be refilled. As shown in FIG. 3A, the diffuser 104 has a fragrance distribution structure 112 configured for placement within the fragrance reservoir 110 of the base 108. In the arrangement shown, the fragrance distribution structure 112 is a single hollow tube through which the fragrance 102 can flow. In other arrangements, the fragrance distribution structure 112 may include a structure having a plurality of conduits or may include a material such as foam configured to facilitate uptake of the fragrance 102. As shown in FIG. 3A, the diffuser 104 further includes an exterior surface 114 fluidly connected to the fragrance distribution structure 112. In particular, the plurality of pores 106 provide the fluid connection between the fragrance distribution structure 112 and the exterior surface 114.

In general, the device 100 is configured to achieve diffusion of the fragrance 102 by evaporation without requiring a power source or electromechanical features. The fragrance 102 to travels from the fragrance reservoir 110 of the base 108, through the fragrance distribution structure 112 of the diffuser 104, through the plurality of pores 106 of the diffuser 104 to the exterior surface 114 of the diffuser 104, and evaporates from the exterior surface 114 of the diffuser. In some arrangements, the diffuser 104 may include a fragrance repository 116 (depicted in FIG. 3A as a removable leaf) that can store fragrance 102 separately from the fragrance reservoir 110 of the base 108 in order to increase an amount of fragrance 102 that can be taken with the diffuser 104 if used independently from the base 108. In such arrangements, the fragrance repository 116 is in fluid communication with the plurality of pores 106 either directly or via the fragrance distribution structure 112.

The device 100 may include features to further facilitate distribution of the fragrance 102. For example, the device 100 may include a heating element 118 as shown in FIG. 2. The heating element 118 may, for example, be heated coils provided around or at the bottom of the fragrance reservoir 110 to heat the fragrance 102, vaporizing at least in part the fragrance 102 in order to facilitate diffusion of the fragrance 102. As another example, the device 100 may include an air circulation device 120. The air circulation device 120 may, for example, be a fan provided along the fragrance distribution structure 112 of the diffuser 104 as shown in FIG. 3A. As yet another example, the device 100 may include a pressure control 122 to create a pressure differential to drive distribution of the fragrance 102. For example, the pressure control 122 may be provided within the base 108 as shown in FIG. 2 in order to create a pressure differential to drive the fragrance 102 from the fragrance reservoir 110 into the fragrance distribution structure 112 and toward the exterior surface 114.

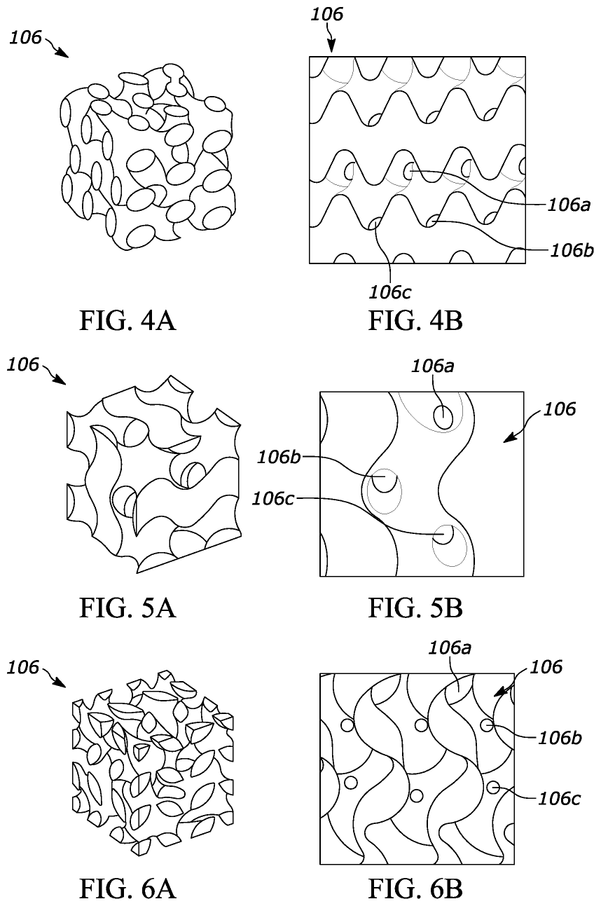

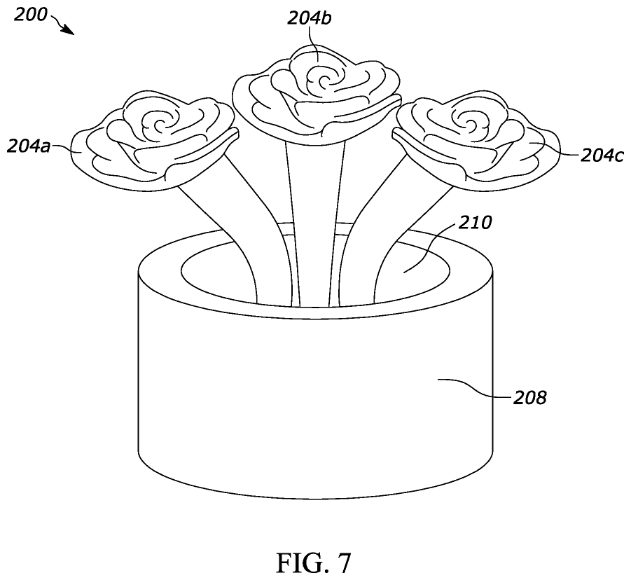

FIGS. 4A-6B show three different porosities of the plurality of pores 106 within the diffuser 104. FIGS. 4A and 4B show a porosity in which the plurality of pores 106 (106a, 106b, 106c . . . as identified in FIG. 4B) are relatively uniform in size, relatively small, and occur at a high frequency. FIGS. 5A and 5B show a porosity in which the plurality of pores (106a, 106b, 106c . . . as identified in FIG. 5B) are relatively uniform in size, relative large, and occur at a low frequency. FIGS. 6A and 6B show a porosity in which the plurality of pores 106 (106a, 106b, 106c . . . as identified in FIG. 6B) vary considerably in size and occur at a relatively middling frequency. Larger and more frequent pores generally contribute to a faster rate of absorption, a greater amount of storage, and faster rate of diffusion, while smaller and less frequent pores generally contribute to a slower rate of absorption, a smaller amount of storage, and a slower rate of diffusion. The viscosity and composition of the fragrance 102 impacts the appropriate porosity of the plurality of pores 106 to achieve a target rate of absorption, amount of storage, and/or rate of diffusion. For example, the concentration of oils, types of oils, and oil particle size impact the appropriate porosity.

FIG. 7 illustrates an additively manufactured device 200 for diffusing a fragrance 202 having a plurality of diffusers 204. Features shared with the device 100 of FIGS. 1-3B are referred to by the same reference number increased by one hundred. After their initial introduction, common features are not described in substantial detail for subsequent arrangements. Unique features are identified by unique reference numbers. Any combination or sub-combination of features described in regard to FIGS. 1-3B may be incorporated into the device 200 of FIG. 7, and vice-versa.

The device 200 of FIG. 7 includes a plurality of diffusers 204 (204a, 204b, 204c) configured for placement within the fragrance reservoir 210 of the base 208. The plurality of diffusers 204 may each have a similar aesthetic, as shown in FIG. 7, or may have a different aesthetic and/or function. For example, aesthetically, the plurality of diffusers 204 may resemble a floral bouquet having different types of flowers. As another example, functionally, some of the plurality of diffusers 204 may be configured to remain permanently within the base 208 while others may be configured to be removed from the base 208 and worn (e.g., as a boutonniere or brooch). The plurality of diffusers 204 may each have an identical porosity or may have differing porosities that allow a user to select a particular porosity for a particular situation. For example, a user may opt to wear a diffuser 204a having a porosity configured for a lower rate of diffusion for a subtle scent on their person while leaving a diffuser 204b within the base 208 having a porosity configured for a higher rate of diffusion in order to provide a higher intensity of scent within the space in which the device 200 is located.

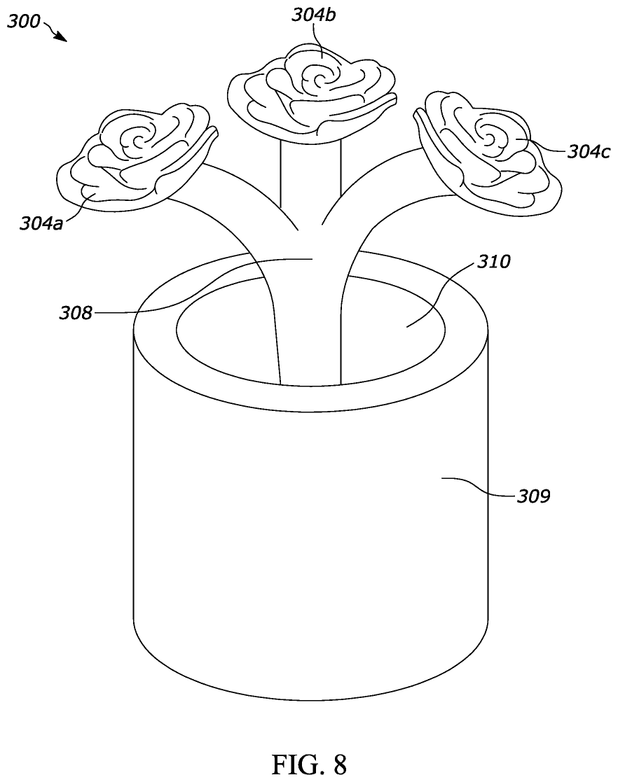

FIG. 8 illustrates an additively manufactured device 300 for diffusing a fragrance 302 having a plurality of diffusers 304. Features shared with the device 100 of FIGS. 1-3B and the device 200 of FIG. 7 are referred to by the same reference number increased by a multiple of one hundred. After their initial introduction, common features are not described in substantial detail for subsequent arrangements. Unique features are identified by unique reference numbers. Any combination or sub-combination of features described in regard to FIGS. 1-3B or FIG. 7 may be incorporated into the device 300 of FIG. 8, and vice-versa. The device 300 of FIG. 8 is similar to the device 200 of FIG. 7 except that the plurality of diffusers 304 (304a, 304b, 304c) are placed in a base 308 that resembles a stem. In the arrangement shown, the base 208 is placed in secondary support 309 that is a vase.

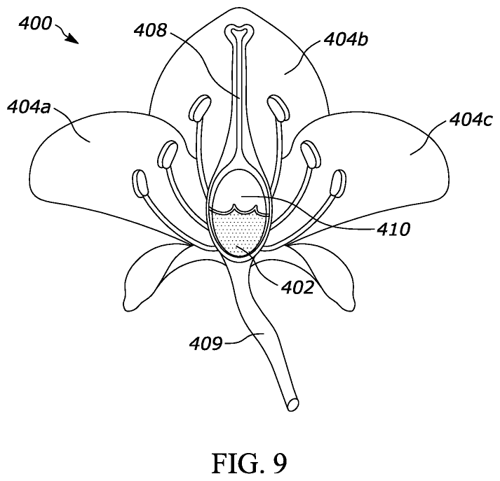

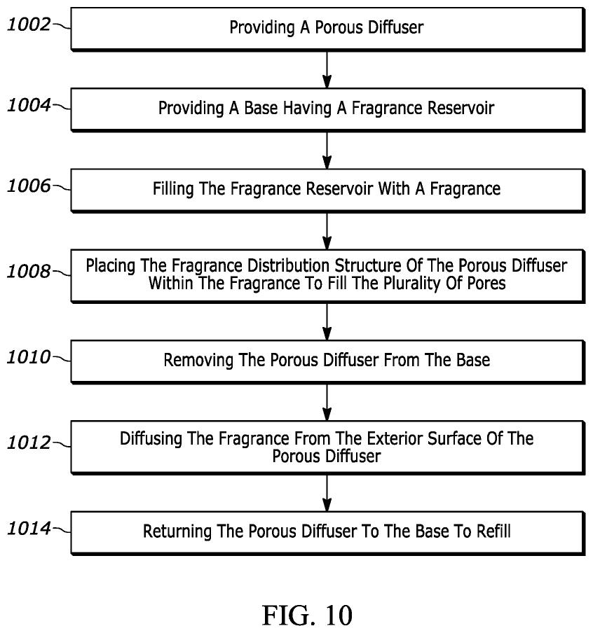

FIG. 9 illustrates an additively manufactured device 400 for diffusing a fragrance 402 having a plurality of diffusers 404. Features shared with the device 100 of FIGS. 1-3B, the device 200 of FIG. 7, and the device 300 of FIG. 8 are referred to by the same reference number increased by a multiple of one hundred. After their initial introduction, common features are not described in substantial detail for subsequent arrangements. Unique features are identified by unique reference numbers. Any combination or sub-combination of features described in regard to FIGS. 1-3B, FIG. 7, or FIG. 8 may be incorporated into the device 400 of FIG. 9, and vice-versa. The device 400 of FIG. 9 is similar to the device 300 of FIG. 8 except that the plurality of diffusers 404 (404a, 404b, 404c) resemble flower petals and are placed in a base 408 that resembles a carpel with the result that the plurality of diffusers 404 and the base 408 together resemble a single flower. In the arrangement shown, the base 408 is placed in a secondary support 409 that resembles a stem. FIG. 10 illustrates a method 1000 of using a porous device, such as the device 100 or 200. At box 1002, the method 1000 includes providing a porous diffuser (such as diffuser 104 or one of the plurality of diffusers 204) having a fragrance distribution structure (such as fragrance distribution structure 112), a plurality of pores (such as the plurality of pores 106), and an exterior surface (such as the exterior surface 114). At box 1004, the method 1000 includes providing a base (such as base 108 or 208) having a fragrance reservoir (such as fragrance reservoir 110). At box 1006, the method 1000 includes filling the fragrance reservoir of the base with a fragrance (such as fragrance 102). At box 1008, the method 1000 includes placing the fragrance distribution structure of the porous diffuser within the fragrance in the fragrance reservoir of the base to fill the plurality of pores of the porous diffuser with the fragrance. At box 1010, the method 1000 includes removing the porous diffuser from the base. At box 1012, the method 1000 includes diffusing the fragrance from the exterior surface of the porous diffuser. At box 1014, the method 1000 includes returning the porous diffuser to the base with the fragrance distribution structure within the fragrance reservoir to refill the plurality of pores of the porous diffuser with the fragrance.

Optionally, the method 1000 may further include refilling the fragrance reservoir with the fragrance. The method 1000 may further include creating a pressure differential (by, for example, a pressure control such as pressure control 122) to drive the fragrance from the fragrance reservoir into the fragrance distribution structure and toward the exterior surface. The method 800 may include providing a visual characteristic within the fragrance that is visible within the porous diffuser when the fragrance is in the plurality of pores. For example, the visual characteristic may be a color such as red.

According to some variations of the method 1000, the degree to which the visual characteristic within the fragrance is visible at the exterior surface (such as the exterior surface 114) of the porous diffuser correlates with the amount of fragrance present within the porous diffuser. For example, the exterior surface may be brighter red when the diffuser is full of fragrance and may slowly fade to a lighter red as the fragrance is diffused and less fragrance remains within the diffuser. According to other variations of the method 1000, a subset of the plurality of pores are configured to trap the fragrance such that the degree to which the visual characteristic within the fragrance is visible at the exterior surface of the porous diffuser increases with use of the porous diffuser as more of the plurality of pores within the subset trap the fragrance. That is, a subset of the plurality of pores may not permit diffusion of the fragrance by, for example, not being located at, near, or in fluid communication with other pores at the exterior surface. Fragrance may become trapped in more and more of such pores over time, resulting in the visual characteristic becoming more pronounced with use of the diffuser.

According to some variations of method 1000, the porous diffuser has a fragrance repository (such as fragrance repository 116) in fluid communication with the exterior surface via the plurality of pores, and the plurality of pores are adapted to transmit the fragrance from the fragrance repository to the exterior surface at a target rate. This increases the duration of time that the porous diffuser can be used before needing to be refilled from the base.

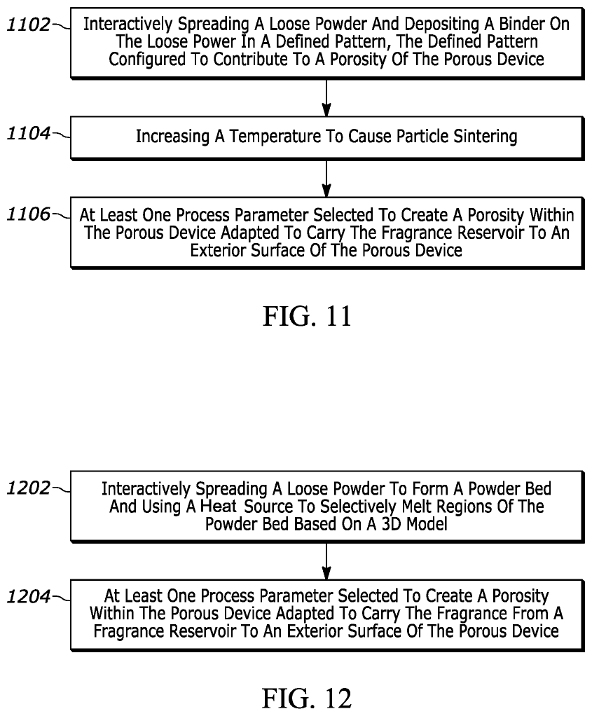

FIG. 11 illustrates a method 1100 of manufacturing a porous device (such as device 100 or 200) using additive manufacturing. At box 1102, the method 1100 includes iteratively spreading a loose powder and depositing a binder on the loose powder in a defined pattern. The defined pattern is configured to contribute to a porosity of the porous device. At box 1104, the method 1100 includes increasing a temperature to cause particle sintering. At box 1106, the method 1100 is defined as having at least one process parameter selected to create a porosity within the porous device adapted to carry the fragrance (such as fragrance 102) from a fragrance reservoir (such as fragrance reservoir 110) to an exterior surface (such as exterior surface 114) of the porous device.

In some variations of method 1100, the at least one process parameter is at least one of a thickness of layers of the loose powder, a viscosity, a surface tension, a solid content, a pH level, a drying rate, an adhesion strength of the binder, a shape of the loose powder, an average particle size of the loose powder, a size distribution of the loose powder, a sintering temperature, and a sintering time. The layer thickness is defined based on the average of the particle size of the loose powder or binder. For loose power, the range can be as low as the average particle size of the loose powder up to as high as 20 times the average particle size of the loose powder. The layer thickness also depends on the shape of the particles. One preferred layer thickness would be six times the average particle size of the loose powder. In some variations of method 900, the loose powder comprises at least one of ceramic, metal, glass, and composites. The laser wavelength may be between 2 and 20 microns. The method 1100 may include saturating the porous device with a fragrance. The method 1100 may include sealing the porous device within an external housing.

FIG. 12 illustrates a method 1200 of manufacturing a porous device (such as device 100 or 200) using additive manufacturing. At box 1202, the method 1200 includes iteratively spreading a loose powder to form a powder bed and using a heat source to selectively melt regions of the powder bed based on a 3D model. The heat source may be a laser source or an infrared source. At box 1204, the method 1200 is defined as having at least one process parameter selected to create a porosity within the porous device adapted to carry the fragrance (such as fragrance 102) from a fragrance reservoir (such as fragrance reservoir 110) to an exterior surface (such as exterior surface 114) of the porous device.

In some variations, the method 1200 has at least one process parameter that is at least one of beam diameter of the heat source, a power of the heat source, a layer thickness, a scanning speed, a powder spreading method, a type of polymer powder, a type of composite powder, a shape of the loose powder, an average particle size of the loose powder, a laser wavelength, a size distribution of the loose powder, and an energy output from an infrared source. The layer thickness is defined based on the average of the particle size of the loose powder or binder. For loose power, the range can be as low as the average particle size of the loose powder up to as high as 20 times the average particle size of the loose powder. The layer thickness also depends on the shape of the particles. One preferred layer thickness would be six times the average particle size of the loose powder. The laser wavelength may be between 2 and 20 microns. The method 1200 may include saturating the porous device with a fragrance (such as fragrance 102).

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising.” “has”, “having,” “includes”, “including.” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A method of manufacturing a porous device for diffusing a fragrance using additive manufacturing, the method comprising:
iteratively spreading a loose powder and depositing a binder on the loose powder in a defined pattern, the defined pattern configured to contribute to a porosity of the porous device; and
increasing a temperature to cause particle sintering,
wherein at least one process parameter is selected to create a porosity within the porous device adapted to carry the fragrance from a fragrance reservoir to an exterior surface of the porous device.

2. The method of claim 1, wherein the at least one process parameter is at least one of a thickness of layers of the loose powder, a viscosity, a surface tension, a solid content, a pH level, a drying rate, an adhesion strength of the binder, a shape of the loose powder, an average particle size of the loose powder, a size distribution of the loose powder, a sintering temperature, and a sintering time.

3. The method of claim 1, wherein the loose powder comprises at least one of ceramic, metal, glass, and composites.

4. The method of claim 1, further comprising saturating the porous device with a fragrance.

5. The method of claim 1, further comprising sealing the porous device within an external housing.

6. A method of manufacturing a porous device for diffusing a fragrance using additive manufacturing, the method comprising:
iteratively spreading a loose powder to form a powder bed and using a heat source to selectively melt regions of the powder bed based on a 3D model,
wherein at least one process parameter is selected to create a porosity within the porous device adapted to carry the fragrance from a fragrance reservoir to an exterior surface of the porous device.

7. The method of claim 6, wherein the at least one process parameter is at least one of a beam diameter of the heat source, a power of the heat source, a layer thickness, a scanning speed, a powder spreading method, a type of polymer powder, a type of composite powder, a shape of the loose powder, an average particle size of the loose powder, a laser wavelength, a size distribution of the loose powder, and an energy output from an infrared source.

8. The method of claim 5, further comprising saturating the porous device with a fragrance.

9. An additively manufactured device for diffusing a fragrance comprising:
a diffuser including a plurality of pores,
the plurality of pores configured to absorb, store, and diffuse a fragrance,
the plurality of pores defining a porosity of the diffuser, and
the porosity of the diffuser defining at least one of a rate of absorption, an amount of storage, and a rate of diffusion of the fragrance.

10. The additively manufactured device of claim 9 further including a base having a fragrance reservoir configured to hold the fragrance, the diffuser including a fragrance distribution structure configured for placement within the fragrance reservoir of the base, the diffuser further including an exterior surface fluidly connected to the fragrance distribution structure.

11. The additively manufactured device of claim 10, a plurality of diffusers configured for placement within the fragrance reservoir of the base.

12. The additively manufactured device of claim 10, the base including a pressure control to create a pressure differential to drive the fragrance from the fragrance reservoir into the fragrance distribution structure and toward the exterior surface.

13. The additively manufactured device of claim 9 further comprising at least one of an air circulation device and a heating element.

14. A method of using a porous device, the method comprising:
providing a porous diffuser having a fragrance distribution structure, a plurality of pores, and an exterior surface;
providing a base having a fragrance reservoir;
filling the fragrance reservoir of the base with a fragrance;
placing the fragrance distribution structure of the porous diffuser within the fragrance in the fragrance reservoir of the base to fill the plurality of pores of the porous diffuser with the fragrance;
removing the porous diffuser from the base;
diffusing the fragrance from the exterior surface of the porous diffuser; and
returning the porous diffuser to the base with the fragrance distribution structure within the fragrance reservoir to refill the plurality of pores of the porous diffuser with the fragrance.

15. The method of claim 14, further comprising refilling the fragrance reservoir with the fragrance.

16. The method of claim 14, further comprising creating a pressure differential to drive the fragrance from the fragrance reservoir into the fragrance distribution structure and toward the exterior surface.

17. The method of claim 14, further comprising providing a visual characteristic within the fragrance that is visible within the porous diffuser when the fragrance is in the plurality of pores.

18. The method of claim 17, wherein the degree to which the visual characteristic within the fragrance is visible at the exterior surface of the porous diffuser correlates with the amount of fragrance present within the porous diffuser.

19. The method of claim 17, wherein a subset of the plurality of pores are configured to trap the fragrance such that the degree to which the visual characteristic within the fragrance is visible at the exterior surface of the porous diffuser increases with use of the porous diffuser as more of the plurality of pores within the subset trap the fragrance.

20. The method of claim 14, wherein the porous diffuser has a fragrance repository in fluid communication with the exterior surface via the plurality of pores, the plurality of pores adapted to transmit the fragrance from the fragrance repository to the exterior surface at a target rate.
